(19) United States
(12) Patent Application Publication
(10) Pub. No.: US 2022/0269763 A1
(43) Pub. Date: Aug. 25, 2022

(54) TAILORING ADMINISTRATION OF AEROSOLIZED BIOACTIVE MATERIAL

(71) Applicant: Medda, Inc., Ira, MI (US)

(72) Inventors: G. Scott Lett, Longmont, CO (US); Jeffrey Humpherys, Lehi, UT (US); Ruth Fisher, Mountain View, CA (US); Chosen Lee, Fort Washington, PA (US); Caleb McClain, Ira, MI (US)

(21) Appl. No.: 17/677,079

(22) Filed: Feb. 22, 2022

Related U.S. Application Data
(60) Provisional application No. 63/151,364, filed on Feb. 19, 2021.

(51) Int. Cl. G06F 21/32; G06F 16/28; A24F 40/53; A24F 40/42; A24F 40/57; A24F 40/65; A61M 11/04

(52) U.S. Cl. CPC G06F 21/32; G06F 16/285; A24F 40/53; A24F 40/42; A24F 40/57; A24F 40/65; A61M 11/041; A61M 2205/6009; A61M 2205/8206; A61M 2205/3341

(57) ABSTRACT

The present invention extends to methods, systems, and computer program products for tailoring administration of aerosolized bioactive material. Aspects include communicating biological and subjective information to support formulation of personalized bioactive material compositions. Aerosolized bioactive materials can be administered to humans, via inhalation, using a vaporizer device which is engineered for real time data capture, and communication, with a software application and backend computational system. Smart vaporizer cartridges can be identified and their contents recalled. Based at least on contents, appropriate (e.g., precision) doses of bioactive material can be aerosolized and administered. Users have the ability to electronically exchange and communicate in real time with the provisioning organization and medical practitioners via telemedicine and can directly participate in clinical trials pertaining to the bioactive material(s) in formulations within the smart vaporizer cartridge and elsewhere.

### CROSS-REFERENCE TO RELATED APPLICATIONS

This application claims the benefit of U.S. Provisional Patent Application Ser. No. 63/151,364, entitled “Tailoring Doses Of Aerosolized Bioactive Material”, filed Feb. 19, 2021, which is incorporated herein in its entirety.

### BACKGROUND

### 1. Background and Relevant Art

Atomizers can be used to deliver bioactive material into a human body. Atomizers can operate as standalone devices or be included in other devices, such as, cartomizers, clearomizer, etc. In general, atomizers turn liquid (e.g., e-liquid, e-juice, etc.) into a fine mist or aerosol for inhalation. A typical atomizer includes: (1) a well or reservoir to hold liquid (e.g., e-liquid, e-juice, etc.), (2) a heating surface to aerosolizer the liquid, (3) a wick to transport the liquid from the reservoir to the heating surface, and (4) a mouth piece where aerosol exits the aerosolizer. Thus, a person can use an atomizer to inhale aerosolized liquid.

Atomizer systems and devices (e.g., stand-alone atomizers or atomizers included in other devices) are usually locally and manually controlled. A person activates atomization and then inhales aerosol. On some atomizer systems and devices, pressing a button activates atomization. On other atomizer systems and devices, detecting inhalation activates atomization. Activating atomization causes liquid to be drawn from the reservoir to the heating surface via the wick. The heating surface then aerosolizes the liquid into aerosol. The aerosol is delivered to a person through a mouth piece.

Typical atomizer systems and devices rely primarily, and in some environments solely, on a user to control aerosol delivery. For example, atomization is controlled by holding down a button or inhaling. A user estimates how much aerosol is inhaled (e.g., a best guess) based on how long a button is depressed and/or how long inhalation lasts. Over time, users may become more skilled at estimating amounts of inhaled aerosol.

However, operating characteristics of atomizer systems and devices can change and/or degrade over time due, at least in part, to using different components and/or component wear and tear. For example, vaporizer pressure can vary when different liquids and/or different liquid reservoirs are used with a heating surface. Further, there is often no way to know the precise contents of liquid being aerosolized, nor the precise volume of aerosol produced for inhalation. Heating surface efficiency can also degrade over time as the heating surface aerosolizes liquid (e.g., resulting in inappropriate temperature variations). Atomizer systems and devices typically lack component monitoring and adjustment mechanisms to account for component variability and/or degradation, or variability of liquid content.

Further, atomizer systems and devices typically include limited, if any, security features. As such, essentially anyone (including children) can activate any atomizer system or device and inhale aerosol.

### BRIEF SUMMARY

Examples extend to methods, systems, and computer program products for tailoring administration of aerosolized bioactive material.

An application at a mobile device (e.g., a smart phone) authorizes vaporizer usage. The vaporizer transmits a product identifier to the application. The application accesses the product identifier and uses the product identifier, potentially via communication with a computational cluster, to identify a bioactive material contained at the vaporizer. The application determines a bioactive material dosage option based at least on bioactive material data corresponding to the identified bioactive material.

The application, potentially via communication with the computational cluster, derives vaporizer commands. The derived vaporizer commands are configured to control the vaporizer and implement the determined dosage option at the vaporizer. The application sends the derived vaporizer commands to the vaporizer.

The vaporizer receives the derived vaporizer commands from the application. The vaporizer implements the vaporizer commands, delivering aerosolized bioactive material external to the vaporizer (e.g., in accordance with the selected dosage option).

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by practice. The features and advantages may be realized and obtained by means of the instruments and combinations particularly pointed out in the appended claims. These and other features and advantages will become more fully apparent from the following description and appended claims, or may be learned by practice as set forth hereinafter.

### BRIEF DESCRIPTION OF THE DRAWINGS

In order to describe the manner in which the above-recited and other advantages and features can be obtained, a more particular description will be rendered by reference to specific implementations thereof which are illustrated in the appended drawings. Understanding that these drawings depict only some implementations and are not therefore to be considered to be limiting of its scope, implementations will be described and explained with additional specificity and detail through the use of the accompanying drawings in which:

FIG. 1 illustrates an example architecture that facilitates tailoring aerosolized doses of bioactive material.

FIG. 2 illustrates a flow chart of an example method for determining contents of a bioactive material holding chamber.

FIG. 3 illustrates a flow chart of an example method for controlling bioactive material delivery.

### DETAILED DESCRIPTION

Examples extend to methods, systems, and computer program products for tailoring administration of aerosolized bioactive material.

In general, aspects of the invention increase accuracy and adjustability of personalized doses of aerosolized bioactive consumable products. Doses of bioactive material can be personalized and continuously adapted, for example, using user-specific data sources, improving personalized bioactive material formulations.

Aspects include real-time data transfer, dosing, control, and analytics via network connectivity between a delivery device a software application, and a computational cluster. A delivery device is hereinafter referred to as a “smart vaporizer device” or simply a “vaporizer device” or “vaporizer”, and which is also defined to include any other atomizer device or system. The software application gathers data from the vaporizer device and transmits to the computational cluster. The software application receives control related data and metrics from the computation cluster and utilizes the control related data and metrics when controlling/adjusting the vaporizer device. The software application can also gather data from additional user-specific sources to correlated the user-specific vaporizer device interaction.

Adjustments to a vaporizer device can include remotely (and potentially automatically) adjusting temperature of a vaporizer device heating element (surface), changing dose, etc. Heating element adjustments can be based at least in part on the composition and appropriate (e.g., optimal) aerosolization temperatures of materials contained in a vaporizer cartridge. Adjustments to a vaporizer device can also include remotely (and potentially automatically) adjusting pressure within a vaporizer cartridge. Pressure can be adjusted based at least in part on a composition of bioactive materials contained within the vaporizer cartridge and an appropriate (e.g., optimum or optimally desired) pressure facilitating a designated, specified, or defined flow rate (e.g., ml/sec) of those materials

The software application and/or computational cluster can be wirelessly connected to the smart vaporizer device. The smart vaporizer device can be mated with (i.e., physically attached to) (or, in some aspects, potentially include) a data encoded vaporizer cartridge (e.g., filled with a formulation of bioactive material). The data encoded vaporizer cartridge can include data encoding and/or components for electronically/digitally indicating the formulation composition, including materials, substances, amounts, percentages, etc. In one aspect, data encoded vaporizer cartridges are uniquely identifiable by a machine-readable code. The smart vaporizer device is configured to read the machine-readable code and transmit the machine-readable code to the software application.

In general, the software application can also remotely identify, monitor, and control usage of the smart vaporizer device and inserted data enabled cartridge combination via the wireless connection. The vaporizer device and the data encoded vaporizer cartridge can transmit usage data to the software application. The software application can gather, monitor, and analyze the usage data. The software application can include machine learning-based code that identifies user-specific vaporizer usage patterns and automatically adjust devices settings to tailor (e.g., optimize) vaporizer device performance and dosage accuracy.

More specifically, the software application can calculate, quantify, and wirelessly adjust vaporizer device settings control a user's intake of active molecules contained within the data encoded vaporizer cartridge. The software application can analyze the gathered usage data, along with device authorization and user-selected dosage settings, to more precisely calculate timing and duration of control commands transmitted to the smart vaporizer device (via wireless communication). Control commands can be used to configured and reconfigure smart vaporizer device components. For example, the software application can implement remote adjustments to the temperature of the vaporizer cartridge heating element and/or pressure inside an encoded cartridge. Temperature and/or pressure adjustments can be automatic and based on the composition and vaporization temperatures of materials contained within a data encoded vaporizer cartridge. Accordingly, aerosolized bioactive material dosing accuracy can be increased and tailored per user.

In one aspect, the application and/or computational cluster automatically identifies data encoded vaporizer cartridges. Each data encoded vaporizer cartridge can be filled with a unique formulation of bioactive material. Bioactive material composition data and cartridge tag registration data may be upload to a computational cluster remotely. Remote upload permits multiple and different manufacturers to contribute data to and recall data from the computational cluster. The molecular analysis data of each formulation contained in the data encoded cartridges is logged and stored in the computational cluster and/or software application. As the application receives identification data from an encoded cartridge, the application accesses specifications of the bioactive material associated with that cartridge. The application utilizes this data to calculate, monitor, and appropriately (e.g., more accurately) control the delivery of aerosolized bioactive material doses generated by the vaporizer device and cartridge.

The application can monitor and gather use metrics and user intake data from a smart vaporizer device. The application may perform analytics on the metrics and/or intake data. The application can transmit data and/or analytics to the computational cluster where it is (e.g., further) analyzed and logged for use in adaptive formulation improvements.

Components included at any of smart vaporizer devices, software applications, and computational clusters can also gather and analyze user-specific data from external sources. External sources can include but not limited to: fitness tracker applications, nutritional tracker applications, biometric wearable devices, biomedical devices, bioinformatics/genomics data, electronic medical records data. A smart vaporizer device, software application, or computational clusters can monitor and/or communicate with external data sources on an ongoing basis. Changes in data from external data sources can be used to evaluate biological circumstances and needs of a smart vaporizer device user. Changes in data from external data sources can be used to identify biological responses induced through vaporizer device use and intake of aerosolizer bioactive material. For example, gathered data can be utilized to continuously adapt and improve formulations of bioactive material.

The application and/or computational cluster can implement protocols facilitating security and safety features that significantly reduce (if not eliminate) any of: unauthorized use of a vaporizer device (e.g., by children), unauthorized use of a mated (e.g., physically attached separate) vaporizer cartridge, use of counterfeit cartridges, use of contaminated cartridges, use of vaporizer when operating a motor vehicle, use of cartridges recalled by a manufacturer, etc. The security and safety features reduce the possibility of children and/or those that might have an adverse reaction to (or are otherwise not intended users of) a formulation from using a vaporizer device and/or a contained vaporizer cartridge. The security and safety features also help ensure accuracy of adaptive formulations by authenticating the user/device interaction. For example, the security and safety features can be used to shut off a smart vaporizer device when a counterfeit or contaminated cartridge is detected.

In one aspect, a smart vaporizer and/or a mobile device software application detects when a cartridge is depleted and records an indication that cartridge is depleted. Upon detecting depletion, the security and safety features can prevent further usage of a depleted cartridge. As such, usage of a smart vaporizer device can be prevented when a detected cartridge was previously detected (and recorded) as depleted. Preventing further user of depleted cartridges mitigates the possibility of cartridges being refilled with unknown material, for example, creating a counterfeit and/or contaminated cartridge.

A smart vaporizer device can include and/or implement controls that check for authentication information from a paired mobile device (e.g., a smart phone). Use of the smart vaporizer device can be prevented when the authentication information is not detected/received. In one aspect, to authorize operation of a smart vaporizer device, a user must enter an elected passcode or biometric passcode (fingerprint scan) using the application.

Accordingly, components for tailoring aerosolized bioactive material doses for a user can include a delivery device (“vaporizer”), cartridges of bioactive product consumed using the delivery device (“cartridge”), a software application running on a remote device (“application”), and a communicating system of computers to store and analyze customer data (“cluster”). The system provides for real-time wireless communication between the components.

Users are able to wirelessly pair a smart vaporizer device to the software application running on their mobile device (e.g., smart phone or tablet) via wireless device-to-device communication (e.g., Bluetooth, wi-fi, D2D 5G, LTE Direct NFC, etc.). Pairing creates a link between the smart vaporizer device and the software application. The smart vaporizer device can transmit sensor data to the software application and receives control commands from the software application. A similar process can be utilized for a smart vaporizer device between a web application and desktop or laptop computer.

The described components can interoperate to facilitate automatic identification of data encoded cartridges (the contents of which are logged into the cluster and are recalled by the software application), recall cartridge identifier data, and simultaneously conduct real-time monitoring and analysis of smart vaporizer device usage. The software application and cluster can utilize cartridge data and smart vaporizer device usage analysis to remotely control the smart vaporizer device and administer doses with increased precision.

The described components can continuously and remotely monitor and gather usage analytics to improve the accuracy of formulations and recommendations based on a user's unique biological, medical, or subjective needs or preferences. The software application and cluster can be programmed to send push notifications and queries to gather pre/post-consumer sentiment regarding a user's current symptoms and severity, as well as provide reminders for the users scheduled dosage intake. Push notifications can also be used to suggest re-ordering of cartridges approaching depletion based on cartridge identifiers.

As described, the software application and cluster can be configured to gather data from third party applications and remote data sources including, but not limited to: biometric wearable devices, bioinformatics and genomics analysis, biomedical records, and sentiment analysis via social media and other public or private sources. The software application and cluster can utilize captured third party data (in combination with smart vaporizer device data and analytics) to continuously adapt and develop more accurate bioactive material formulations, and to provide greater accuracy of dose and activity recommendations based on a user's evolving needs or intended use(s).

Adjustments to heating element temperature can be configured using the application. The application can facilitate remote manual adjustment of heating element temperature (e.g., through a user interface). A user can enter a temperature adjustment into the application and the application can implement the temperature adjustment at the smart vaporizer device over the paired link. The application can also implement automated heating element temperature adjustments via the paired link. Adjustments to heating element temperature can be based on the composition and aerosolization temperatures of materials contained within an identified data encoded cartridge and its associated formulation of bioactive materials. Appropriate heating element temperature can help ensure and optimize continuous flow and aerosolization of contained bioactive materials based on material composition. Appropriate heating element temperature can also increase precision of aerosolizing bioactive materials based on a material composition.

Vaporizer cartridges can be engineered with a pressure control designed to pressurize and de-pressurize the cartridge to various states of pressurization. Adjustments to vaporizer cartridge pressure can also be configured using the application. The application can facilitate remote manual adjustment of cartridge pressure (e.g., through a user interface). A user can enter a pressure adjustment into the application and the application can implement the pressure adjustment at the smart vaporizer device over the paired link. The application can also implement automated cartridge pressure adjustments via the paired link. Appropriate cartridge pressurization ensures and optimizes continuous flow and aerosolization of contained bioactive materials based on material composition. Appropriate cartridge pressurization can also increase precision of aerosolizing bioactive materials based on a material composition.

In this description and the following claims, “aerosol” is defined as a suspension of fine solid particles or liquid droplets in air or another gas. In one aspect, liquid or solid particles have diameters of less than 1 μm.

Implementations can comprise or utilize a special purpose or general-purpose computer including computer hardware, such as, for example, one or more computer and/or hardware processors (including any of Central Processing Units (CPUs), and/or Graphical Processing Units (GPUs), general-purpose GPUs (GPGPUs), Field Programmable Gate Arrays (FPGAs), application specific integrated circuits (ASICs), Tensor Processing Units (TPUs)) and system memory, as discussed in greater detail below. Implementations also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations can comprise at least two distinctly different kinds of computer-readable media: computer storage media (devices) and transmission media.

Computer storage media (devices) includes RAM, ROM, EEPROM, CD-ROM, Solid State Drives (“SSDs”) (e.g., RAM-based or Flash-based), Shingled Magnetic Recording (“SMR”) devices, Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

In one aspect, one or more processors are configured to execute instructions (e.g., computer-readable instructions, computer-executable instructions, etc.) to perform any of a plurality of described operations. The one or more processors can access information from system memory and/or store information in system memory. The one or more processors can (e.g., automatically) transform information between different formats, such as, for example, between any of: product identifiers, bioactive material data, formulation data, user/profile data, cartridge ID data, vaporizer control commands, dosage data, sensors data, bioactive material inhalation data, vaporizer operational data, etc.

System memory can be coupled to the one or more processors and can store instructions (e.g., computer-readable instructions, computer-executable instructions, etc.) executed by the one or more processors. The system memory can also be configured to store any of a plurality of other types of data generated and/or transformed by the described components, such as, for example, product identifiers, bioactive material data, formulation data, user/profile data, cartridge ID data, vaporizer control commands, dosage data, sensors data, bioactive material inhalation data, vaporizer operational data, etc.

In one example, device local memory can enable gathering of device usage data independent from a linked software application for a duration of time relative to the volume of device memory. This usage data is then uploaded to the software/mobile device memory upon next wireless connection. An LED-backed activation button on the device will flash or change color to prompt that device memory has become full and user should pair the device to upload memory.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that computer storage media (devices) can be included in computer system components that also (or even primarily) utilize transmission media.

Computer-executable instructions comprise, for example, instructions and data which, in response to execution at a processor, cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Aspects of the invention may be practiced in network computing environments with many types of computer system configurations, including, personal computers, desktop computers, laptop computers, message processors, hand-held devices, wearable devices, multicore processor systems, multi-processor systems, microprocessor-based or programmable consumer electronics, network PCs, minicomputers, mainframe computers, mobile telephones, PDAs, tablets, routers, switches, smart vaporizing devices, computational clusters, databases, and the like. The described aspects may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Further, where appropriate, functions described herein can be performed in one or more of: hardware, software, firmware, digital components, or analog components. For example, one or more Field Programmable Gate Arrays (FPGAs) and/or one or more application specific integrated circuits (ASICs) and/or one or more Tensor Processing Units (TPUs) can be programmed to carry out one or more of the systems and procedures described herein. Hardware, software, firmware, digital components, or analog components can be specifically tailor-designed for a higher speed processing or artificial intelligence that can enable processing. In another example, computer code is configured for execution in one or more processors, and may include hardware logic/electrical circuitry controlled by the computer code. These example devices are provided herein purposes of illustration, and are not intended to be limiting. Embodiments of the present disclosure may be implemented in further types of devices.

The described aspects can also be implemented in cloud computing environments. In this description and the following claims, “cloud computing” is defined as a model for enabling on-demand network access to a shared pool of configurable computing resources. For example, cloud computing can be employed in the marketplace to offer ubiquitous and convenient on-demand access to the shared pool of configurable computing resources (e.g., compute resources, networking resources, and storage resources). The shared pool of configurable computing resources can be provisioned via virtualization and released with low effort or service provider interaction, and then scaled accordingly.

A cloud computing model can be composed of various characteristics such as, for example, on-demand self-service, broad network access, resource pooling, rapid elasticity, measured service, and so forth. A cloud computing model can also expose various service models, such as, for example, Software as a Service (“SaaS”), Platform as a Service (“PaaS”), and Infrastructure as a Service (“IaaS”). A cloud computing model can also be deployed using different deployment models such as private cloud, community cloud, public cloud, hybrid cloud, and so forth. In this description and in the following claims, a “cloud computing environment” is an environment in which cloud computing is employed.

Architecture

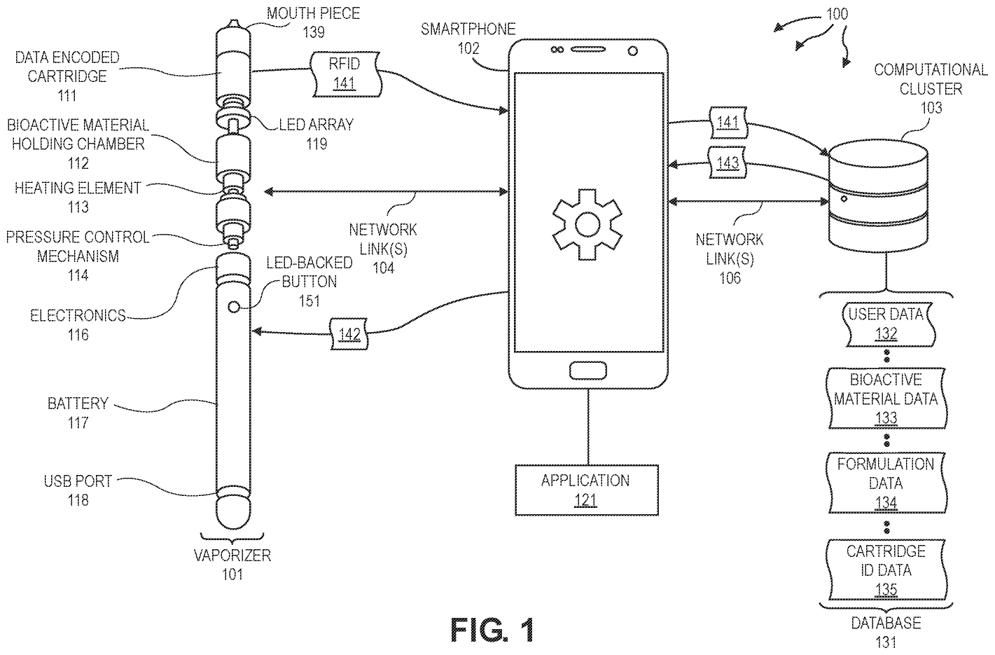

FIG. 1 illustrates an example architecture 100 that facilitates tailoring aerosolized doses of bioactive material. As depicted, architecture 100 includes vaporizer 101, smartphone 102, and computation cluster 103. In one aspect, computational cluster 103 includes cloud-based computing resources. Vaporizer 101 and smartphone 102 are connected via network link(s) 104. Smartphone 102 and computational cluster 103 are connected via network link(s) 106.

In some aspects, a separate data encoded cartridge 111 is mated with (i.e., physically attached to via matching threads) vaporizer 101. In other aspects, vaporizer 101 includes data encoded cartridge 111. Thus, vaporizer 101 and/or encoded cartridge 111 can include: bioactive material holding chamber 112 (e.g., holding a formulation of bioactive material), heating element 113, pressure control mechanism (e.g., valve) 114, and LED array 119. Vaporizer 101 can further include electronics 116, battery 117 (e.g., a lithium-ion battery), USB (e.g., charging/data) port 118, and LED-backed button 151. A mouthpiece 139 can be attached to data encoded cartridge 111. Smart phone 102 further includes application 121. Computational cluster 103 further includes user data 132, bioactive material data 133, formulation data 134, and cartridge ID data 135 stored in database 131. Bioactive material data 133 and/or formulation data 134 can indicate amounts of bioactive material in milligrams, micrograms, or milliliters.

Smartphone 102 can include a touch screen and host applications, including application 121, that communicate via network links 104 with components of vaporizer 101 (e.g., data encoded cartridge 111 or other components included in electronic 116). Smartphone 102 can support various wireless protocols including one or more of: Bluetooth, Radio Frequency (RF), WiFi, etc. Smartphone 102 can communicate with vaporizer 101 using any supported wireless protocol, including accessing class/type data associated with and/or embedded in data encoded cartridge 111 from vaporizer 101. Smartphone 102 can be enabled for push notifications and/or queries displayed on the touch screen, where a user can interact with application 121 (or other applications) and receives usage recommendations, messages, suggestions, reminders, etc.

Vaporizer 101 can support various wireless protocols including one or more of: Bluetooth, RF, WiFi, etc. Vaporizer 101 can communicate with smartphone 102 using any supported wireless protocol, including transmitting class/type data associated with and/or embedded in data encoded cartridge 111 to smartphone 102. In one aspect, electronics 116 further includes a processor, system memory, durable storage, a Radio Frequency (RF) transmitter, and a Bluetooth module. In general, battery 117 provides electrical power to other components of vaporizer 101.

When data encoded cartridge 111 is attached (e.g., mated) to vaporizer 101, (e.g., a processor in) electronics 116 can send an interrogatory signal to a (e.g., RF or data storage) component contained in/or on data encoded cartridge 111. The component can return RFID 141 (an identifier of data encoded cartridge 111) back to electronics 116. An RF transmitter can then transmit RFID 141 to smartphone 102 using a device authorization protocol. Smartphone 102 can deliver RFID 141 to application 121. In response to receiving RFID 141 various components at vaporizer 101 and smartphone 102 can be paired to one another. Vaporizer 101 can then begin to send real-time vaporizer device usage data to software application 121 via network link(s) 104 (e.g., using a Bluetooth protocol).

Application 121 can control vaporizer 101 using various functionality protocols. Application 121 can send vaporizer control commands 142 to vaporizer 101 (e.g., via Bluetooth) after authorization. Vaporizer 101 can receive vaporizer control commands 142 from smartphone 102. Vaporizer control commands 142 can include run commands instructing vaporizer 101 to continue aerosolizing material contained in bioactive material holding chamber 112. Vaporizer control commands 142 can also include stop commands instructing 101 to stop aerosolizing material contained in bioactive material holding chamber 112. Application 121 can send a stop command when a calculated dosage is approaching or has reached a dosage threshold.

Vaporizer 101 can respond to control commands accordingly. For example, a processor in electronics 116 can permit aerosolization in response to run commands by translating a run command into an “on” command for turning battery 117 “on”. On the other hand, the processor in electronics 116 can prevent aerosolization in response to stop commands by translating a stop command into an “off” command for turning battery 117 “off”.

Vaporizer control commands can be derived using a quantitative molecular administering protocol. The quantitative molecular administering protocol can utilize instance metrics, including temperature of smart vaporizer cartridge heating element, rate of aerosolization of the contents of the smart vaporizer cartridge, duration of smart vaporizer device use, and vaporizer flowrate to quantify a user's intake, in real time. As a designated dosage of bioactive material (from bioactive material holding change 112) is being administered, application 102 can send “run” commands to vaporizer 101. In response, vaporizer 101 continues to permit aerosolization (e.g., turning or leaving battery 117 “on”). A “run” command can also be sent when authorization succeeds at application 121, for example, in response to entry of a valid passcode. When a designated dose is administered, application 102 can send a “stop” command. In response, vaporizer 101 prevents further aerosolization (e.g., turn or leave battery 117 “off”). A “stop” command can also be sent when authorization fails at application 121, for example, when an incorrect passcode is entered.

Vaporizer control commands 142 can also include commands to change the temperature of heating element 113 or pressure of data encoded cartridge 111. In one aspect, a processor in electronics 116 controls the amount of voltage (e.g., the strength of current) sent to heating element 113. Variable strength of electrical current generated by the battery 117 is utilized by device in adjusting temperature of the heating element 113 and other device controls.

Vaporizer

As described, vaporizer 101 can include data encoded cartridge 111, bioactive material holding chamber 112 (e.g., holding a formulation of bioactive material), heating element 113, pressure control mechanism (e.g., valve) 114, electronics 116, battery 117 (e.g., a lithium-ion battery), and USB (e.g., charging/data) port 118. Smart phone 102 further includes application 121. Computational cluster 103 further includes user data 132, bioactive material data 133, formulation data 134, and cartridge ID data 135 stored in database 131.

In some aspects, bioactive material holding chamber 112 is constructed of: thermal rated resin or composite, borosilicate glass, or similar materials.

In general, heating element 113 (e.g., a core and coil) receives an electrical or magnetic signal from battery 117 to facilitate aerosolization of bioactive materials contained in bioactive holding chamber 112. Heating element 113 can include a semiconductor, ceramic film, ceramic coil, metal film, thick film heaters, electromagnetic coils, polymer wrapping, polymer PTC, glass-wrapped tungsten or other metal filament, etc. that heats up and aerosolizes bioactive materials. Composition of a heating element can be selected to reduce off gassing. The temperature of heating element 113 is adjustable and can be controlled remotely by application 121. Temperature of hearing element 113 can be configured based on composition(s) of bioactive materials and the optimum aerosolization temperature(s) of those materials.

In some aspects, heating element 113 receives an electrical current signal from battery 117. The electrical current causes the temperature of heating element 113 to rise. Through one or more of convection, conduction, or combustion, the increase in heat causes bioactive material in bioactive material holding chamber 112 to aerosolize. Heating element 113 can be adjustable to different discrete temperatures or to any temperature within a temperature range. The temperature of heating element 113 can be controlled by application 121. Electrical current supplied to heating element 113 can be increased to raise the temperature of heating element 113. On the other hand, electrical current supplied to heating element 113 can be decreased to lower the temperature of heating element 113.

Accordingly, heating element 113 can be engineered as a component that heats bioactive material in bioactive material chamber 112 (e.g., to variable temperatures based on the composition of the material(s)) converting the bioactive material from, for example, lipid or oil form, to aerosol that can be inhaled. The aerosol contains bioactive molecular content found in the original lipid/oil material form. As vaporizer 101 is activated and during use, battery 117 conducts a current to a (e.g., semiconductor) component within data encoded cartridge 111. In turn, heating element 113 is activated, increasing in temperature and (at a above a threshold temperature) converting bioactive material to aerosol. Application 121 and/or computational cluster 103/database 131 can facilitate remote manual temperature adjustments of the heating element 113 temperature as well as automatic remote temperature adjustments of heating element 113 based on the composition and vaporization temperatures of materials contained bioactive material holding chamber 112.

Pressure control mechanism 114 is configured to pressurize and de-pressurize data encoded cartridge 111 to various states of pressurization, based at least in part on a composition of bioactive materials and an appropriate (e.g., optimum) pressure required to ensure flow of materials with those composition(s).

In one aspect, bioactive holding chamber 112, heating element 113, pressure control mechanism 114, LED array 119, and mouth piece 139 are integrated into data encoded cartridge 111. Data encoded cartridge 111 can be configured to accept and aerosolize lipid, oil, resin, dry flower, or other material forms. Data encoded cartridge 111 can aerosolize materials across a range of forms, as well as lipid/oil viscosities.

Data encoded cartridge 111 can be labeled or engineered with an embedded (e.g., RFID or NFC) product recognition tag or machine-readable code (e.g., QR). When data encoded cartridge 111 is inserted into vaporizer 101, the product recognition tag is utilized for instance identification (e.g., by a processor in electronics 116). Data encoded cartridge 111 can be powered by battery 117. When powered, the product recognition tag can emit a (e.g., unique) frequency/data (e.g., via RF or Bluetooth). Application 121 can use the frequency/data to identify data encoded cartridge 111 (e.g., by inventory number, possibly included in cartridge ID data 135).

Emitted data can pertain to qualitative and quantitative measurements of contents within bioactive material holding chamber 112. Application 121 can use the emitted data to enact control settings related to the duration of appropriate dosages (e.g., control commands 142), set appropriate (e.g., optimum) pressure within data encoded cartridge, and set appropriate temperature of the heating element to achieve desired aerosolization.

In general, a product recognition tag is either affixed to or embedded in data encoded cartridge 111. Encoded data can include identification data, including date and time of manufacture and/or preparation of contents in bioactive material holding chamber 112, which corresponds to the unique quantitative formula of bioactive material. As data encoded cartridge 111 is inserted into vaporizer 101, the data encoded identifier tag receives an interrogatory signal from the RF transceiver in electronics 116. The data encoded identifier tag returns encoded data (e.g., RFID 141) to the RF transceiver, which is relayed via network links 104 to application 121. Application can use the encoded data to identify the contents of bioactive material holding chamber 112 and an intended user.

In some aspects, a product recognition tag is an RFID or NFC tag or label. The product recognition tag can operate in a High-Frequency MHz range. A product recognition tag can comply with NFC-V Technology specifications from the NFC Forum (e.g., type 5 tags or labels). Data from a product recognition tag can be transferred to smartphone 102 or other NFC/RFID HF readers that support the NFC Forum standardized NFC Data Exchange Format (NDEF). The radio frequency identification data is associated with a specific instance of a formulation of bioactive material contained within bioactive material holding chamber 114.

Data encoded cartridge 111 can include mouth piece 139, for example, designed for an ergonomic fit in between a user's lips. In some aspects, mouth piece 139 is constructed from ceramic, plastic, or thermal rated resin. Mouthpiece 139 may be an active component of vaporizer 101 and may include LED array 119. LED array 119 can detect aerosol flow rate and primary bioactive aerosol particles (PBAPs) and transmit such data to a processor in electronics 116 and/or application 121.

Accordingly, in some aspects, data encoded cartridge 111 includes a radio transponder and a radio receiver and transmitter. A product recognition tag is powered by energy from the RFID reader's interrogating radio waves. A product recognition tag may be a microchip connected to an antenna. The antenna can be made out of copper, aluminum, or silver strips. The antenna can take different shapes, including: a spiral, a single dipole antenna, two dipoles with one dipole perpendicular to another, or a folded dipole. The antenna length and geometry can be based at least in part on the frequency at which the product recognition tag operates.

The microchip and antenna can be embedded onto a (e.g., thin plastic) substrate of 100 to 200 nm, for example, polymer, PVC, (PET), phenolics, polyesters, styrene, or paper via copper etching or hot stamping. The substrate is attached to or embedded in data encoded cartridge 111.

As described, electronics 116 can include a Bluetooth module. The Bluetooth module can communicate using Bluetooth protocols with a corresponding Bluetooth module at smartphone 102 to facilities data transfer between vaporizer 101 and smartphone 102. Bluetooth module can exchange data (e.g., user intake metrics) with smartphone 102 as well as receive control commands from smartphone 102.

In one aspect, a Bluetooth module includes two parts which may or may not be physically separate: a radio device responsible for modulating and transmitting the signal and a digital controller. The digital controller runs a link controller interfacing vaporizer 101. The link controller is responsible for processing of the baseband and the management of ARQ and physical layer FEC protocols. In addition, the link controller handles the transfer functions (both asynchronous and synchronous), media coding (e.g., SBC (codec)) and data encryption.

A processor in electronics 116 can have responsibility for attending the instructions related to Bluetooth of the host device in order to simplify its operation. To do this, the CPU runs software called Link Manager that has the function of communicating with other devices through the LMP protocol. The Link Manager (LM) is the system that manages the establishment of the connection between devices. It is responsible for the establishment, authentication and configuration of the link. The Link Manager locates other managers and communicates with them via the management protocol of the LMP link. To perform its function as a service provider, the LM uses the services included in the Link Controller (LC). The Link Manager Protocol basically consists of several PDUs (Protocol Data Units) that are sent from one device to another.

As described, electronics 116 can also include an RF transceiver. The RF transceiver can receive serial data from the data encoded identifier tag embedded in data encoded cartridge 111. The RF transceiver can wirelessly transmit interrogatory signals to the product recognition tag through an antenna. The transmitted data is also received by an RF receiver protocol within smartphone 102, which can operate at the same frequency as that of the transmitter.

In one aspect, the RF transceiver includes a circuit designed for half-duplex or full-duplex operation. a transmitter module may be implemented alongside a microcontroller which provides data to the module which can be transmitted. Super-heterodyne, or receivers, are implemented to receive the modulated RF signal and demodulate it. An SoC (System on a Chip) module may also be implemented and is similar to a transceiver module, but can be made with an onboard microcontroller. The microcontroller can be used to handle radio data packetization or to manage a protocol such as an IEEE 802.15.4 compliant module. The RF transceiver module may be useful at least in part due to additional processing for compliance with a protocol when the designer does not wish to incorporate this processing into another processor in electronics 116.

A wide range of different bioactive materials suitable for aerosolizing can be contained in bioactive material holding chamber 112. Bioactive materials can include a unique quantitative formula of active molecules, based on an identified therapeutic treatment pathway and personalized based on a specific user's health condition, or formulated for a standardized targeted desired biological effect of the material. As the formulation is prepared, a product recognition tag is coded with data pertaining to the specific formulation of contained materials. The product recognition tag is then embedded on/into data encoded cartridge 111 and can later be identified by vaporizer 101 and/or smartphone 102.

Formulation data pertaining to product recognition tags (including RFID 141) can be stored and collated in formulation data 134. As such, computational cluster 103 can perform essentially continuous analytics collating and formulae efficacy evaluation. The results of analytics collating and formulae efficacy evaluation can be sent to application 121 via network link(s) 106.

Bioactive materials can include cannabinoids, terpenoids, psilocybin, (dimethyl) tryptamines, flavonoids, alkaloids, phenylethylamines, and/or any other plant or fungus material extract. Bioactive materials can also include inorganic or synthetic compounds, mixtures, and molecules.

Bioactive materials can include refined plant-, herb-, and fungus-derived extracts. Refinement of plant-, herb-, and fungus-derived extracts can implement an extraction process including, but not limited to, subcritical or supercritical CO2 extraction, ethanol extraction, wiped film distillation, column chromatography, or other forms of fractional molecular distillation. Refinement processes can be utilized to separate bioactive molecular content of the original raw material, then purify, concentrate, and isolate the individual molecules or compounds for measurement and dosage in the preparation of formulations.

Other examples of bioactive materials that may be refined and/or contained in bioactive material holding chamber 112 include, but are not limited to, terpenoids, flavonoids, plant extracts, cannabinoids (CBD, THC, CBN, CBG, etc.), fine research chemicals, refined or synthetic organic substances including but not limited to: crystallized psilocybin; 4-Acetoxy-N,N-dimethyltryptamine; N,N-dimethyltryptamine; 5-methoxy-N,N-dimethyltryptamine; 4-AcO-DMT; 3,4,5-Trimethoxyphenethylamine; 2-CB; plant extracts; fungi extracts; lipids; salts; combinations thereof or any other bioactive material (including those developed after the filing date of this application). Selected bioactive materials may have therapeutic biological effects or may show promising therapeutic effects and/or may have been identified for use within a clinical research setting, or commercially as well as combinations thereof.

Bioactive materials can undergo quantitative analysis, to identify the concentrations of any/all bioactive and/or non-bioactive molecules present within the formulation. Quantitative analysis results can be stored in formulation data 134 (e.g., at or near the time of manufacture).

In general, battery 117 can power other components of vaporizer 101 including heating element 113, pressure control mechanism 114, electronics 116 (including a processor and communication modules), and LED array 119.

Battery 117 can include an electrical connection to USB port 118. As such, battery 117 can also power external devices connected to vaporizer 101 at USB port 118. Further, USB port 118 can be used to charge battery 117. To charge battery 117, USB port 118 can be connected to an external power supply or wall outlet. Battery 117 can be designed for cross-compatibility with third party manufactured smart vaporizer cartridges which are also embedded with data encoded tags or labels. An onboard central processing unit included in electronics 116 can decode, process, receive, and transmit data via a Bluetooth module between vaporizer device 101 and smartphone 102.

In one aspect, battery 117 is configured to output a variable voltage and electrical current output range between 3.0 v-5.0 v, for 900 Mah (milliamp hours) or above.

USB port 118 can including virtually any type of USB connection such as, for example, a USB type A, A-micro, or A-mini; B, B-micro, or b-mini; or class-C connector/cable. Variations in the data transfer rate of this component may include USB v1, v2, or v3 with USB data transfer of Low Speed, Full Speed, High Speed (from version 2.0 of the specification), SuperSpeed (from version 3.0), and SuperSpeed+(from version 3.1). USB modes have differing hardware and cabling requirements.

USB Port 118 also permits a processor in electronics 116 to receive a wired transmission from an external data source, for purposes of external controls, data transfer, software, or device firmware updates and/or upgrades. USB port 118 may also provide a link to an external hardware data protocols module, which contains the RF transceiver capabilities, firmware, and/or software necessary to transmit data from vaporizer 101 to application 121. A wired data transfer solution may be a supplement or replacement for an RF transceiver included in electronics 116.

Accordingly, USB port 118 can also provide a link to external hardware and data protocols, which contain RF transceiver capabilities, firmware, and/or software necessary to transmit data from vaporizer 101 to application 121. A wired data transfer solution may be a supplement or replacement for the RF transceiver component within vaporizer 101. Vaporizer 101 and battery 117 may have multiple USB ports, for compatibility with multiple charging, data, or smartphone devices.

As described, electronics 116 can include a processor. Among other functions, the processor can be a computational component configured to facilitate communication and data decoding data transmissions. The processor can convert run/stop protocol commands into on/off commands. The processor can send the on/off commands and discrete voltage output commands via electrical signal to battery 117.

In one aspect, electronics 116 can include an RF transceiver/Bluetooth interface. The RF transceiver/Bluetooth interface can include a combination of: an RF Transceiver, a Bluetooth module component, multiple USB ports, etc. The multiple USB ports can accommodate USB-mini, USB-micro, or USB-C connectivity, etc. The RF Transceiver, a Bluetooth module component, multiple USB ports, possibly along with a processor can be contained within a shell casing.

An RF transceiver/Bluetooth interface can alternatively perform tasks otherwise described in (e.g., RFID) protocols implemented by application 121. In some aspects, the RF transceiver/Bluetooth interface is an external module connected to USB port 118. Thus, if smartphone 102 is not equipped with appropriate RFID protocols sufficient to transmit or receive data and communicate said data to/from application 121, vaporizer 101 can perform functions as intermediary or auxiliary remote interface between smartphone 102 and vaporizer 101 (in some aspects via a connected external hardware module).

In another aspect, a separate external RFID/Bluetooth device interface is insertable into USB port 118 or a USB port of smartphone 102. The external RFID/Bluetooth device is phantom-powered by either the smartphone battery or battery 117 when inserted. The external RFID/Bluetooth device can include multiple physical USB ports, such as, USB-mini, USB-micro, and USB-C, making it cross-compatible with various smartphones (and thus expanding USB port 118 to multiple ports). A connection to smartphone 102 or vaporizer 101 is facilitated using an appropriate USB—mini, USB-micro, or USB-C cable, which creates a wired “tether” The tether provides a data transmission channel through which vaporizer 101, smartphone 102, and application 121 can exchange data.

LED array 119 can include of a compact array of light emitting diodes (LED). LED array 119 can optically indicate aerosol flow related and enable fluorescence-based (optical) detection of single flowing bioaerosols, or primary bioactive aerosol particles (PBAPs). LED array 119 can be included in mouth piece 139 or data encoded cartridge 111. Appropriate electrical connections can connect LED array 119 to battery 117.

LED-backed button 151 can operate as both an indicator and a manual control button. LED-backed button 151 can include a multi-color LED. A processor in electronics 116 can change the color of the multi-color LED and/or turn the multi-color LED on and off in different patterns to indicate various status of vaporizer 101. Indicated status can include when vaporizer 101 is in use, when vaporizer 101 is disabled, when data encoded cartridge 111 is nearing depletion, when battery 117 needs to be recharged, when memory in electronics 116 is nearly full, etc.

In one aspect, LED-backed button 151 includes a small touch screen sensor, configured to detect a thumb print or finger print. The detected thumb print or finger print may function as the passcode. Vaporizer 101 can send the detected thumb print or finger print to application 121 for verification/validation. When the detected thumb print or finger print is verified/validated, application 121 can authorize use of vaporizer 101. Application 121 may access a previously stored thumb print or finger print from user data 132 for verification/validation against a detected thumb print or finger print received from vaporizer 101.

Vaporizer 101 can implement a master/slave protocol. A Bluetooth module at vaporizer 101 (either in electronics 116 or within the insertable RFID/Bluetooth device interface) can include an organization unique identifier (OUI). The OUI can be used by the Bluetooth protocols at smartphone 102 to identify vaporizer 101. At smartphone 102, a user can user the OUI to sync smartphone 102 with vaporizer 101. Syncing can establish a connection with smartphone 101 as the “master” and vaporizer 101 as the “slave”. It some aspects, establishment of a master/slave connection enables application 121 to implement various control functions to control vaporizer 101. In other aspects, application 121 controls vaporizer 101 without establishment of a master/slave connection.

In some aspects, vaporizer 101 also includes sensors that monitor the status and operation of various vaporizer components. For example, a temperature sensor can monitor the temperature of heating element 113. A pressure sensor can monitor the pressure in data encoded cartridge 111. One or more other sensors, for example1 LED array 119, can monitor and optically indicate a volume of aerosol exiting data encoded cartridge 111 (e.g., being inhaled) through mouth piece 139. Data from vaporizer sensors can be sent to processor 116 smartphone 102/application 121 via (wireless or wired) network link(s) 104. Algorithms at application 121 can use the sensor data when determining how to control or manage vaporizer 101. Sensor data can be used along with other described data types when making vaporizer control or management determinations.

Data encoded cartridge 111 can be an externally attached component. One end of vaporizer 101 can include a “male” thread pattern. The “male” thread pattern can be configured to match a corresponding “female” thread pattern of data encoded cartridge 111. Alternatively, vaporizer 101 can include “female” threads and data encoded cartridge 111 can include “male” threads. Data encoded cartridge 111 can include bioactive material holding chamber 112, heating element 113, a data encoded tag, and a mouth piece.

Vaporizer Control Application

As described, smartphone 102 can include a touch screen and application 121. In general, application 121 can be include software for managing and controlling vaporizer 101.

Application 121 can be configured to perform a variety of vaporizer related functions. Application 121 can operate as a “hub” for incoming/outgoing data to/from vaporizer 101, for example, form external user wearables, applications, digital health tools, etc. Application 121 can also perform both discrete and continuous tasks and functions which control vaporizer 101 and calculate usage data associated with vaporizer 101. Application 121 can communicate with vaporizer 101 via communication link(s) 104 to access a product recognition tag associated with date encoded cartridge 111. Application 121 can include a vaporizer device authorization safety protocol, utilizing a passcode or biometric data to activate battery 117.

Application 121 can include a dosage calculation protocol/algorithm that determines when a user has inhaled a designated dose of bioactive material. Application 121 can include run/stop functionality sending control commands 142 to vaporizer 101 as appropriate. Application 121 can implement a quantitative molecular administering protocol. Application 121 can implement any of queries, a push notifications protocol, or a predictive refill reminder push notification protocol. Application 121 can calculate the appropriate single instance and daily dosage which has been determined appropriate or desired by a user. All processes and capabilities available in application 121 can alternatively be implemented in a desktop web application platform.

Application 121 can implement a remote data transfer protocol. The remote data transfer protocol can be used to transmit, receive, decode, and error correct data transferred between the vaporizer 101 and software application 121. The remote data transfer protocol can be implemented using formats such as XML (eXtensible Markup Language) or JSON (Java Script Object Notation). The remote data transfer protocol provides a unified communications language for components at different devices to communicate and to transmit and receive data and commands, for example, via a Bluetooth connection established between the Bluetooth module, an RFID transceiver, and an RFID identification protocol.

Application 121 can implement a device authorization and user/child safety protocol. The device authorization and user/child safety protocol can include a set of programming commands enabled when a user enters an elected user-specific passcode. For example, a passcode entry in the form of an alphanumeric and/or biometric (fingerprint) scan can be used to engage vaporizer 101. The device authorization and user/child safety protocol provides a child-safety feature, preventing unauthorized or unmonitored use of vaporizer 101. The device authorization and user/child safety protocol also provides advanced user identification helping ensure ongoing accuracy of user-specific data received from vaporizer 101. Application 121 can also be linked to a Global Positioning System (GPS) features of smartphone 102. Application 121 may restrict use of vaporizer 101, when GPS detects a user moving in excess of normal walking speed (e.g., greater than 5 miles per hour) or potentially operating a motorized vehicle.

Application 121 can implement a dosage calculation protocol. The dosage calculation protocol can include one or more calculation protocols using specifications of vaporizer 101, such as, as flow rate (e.g., ml/sec), heating element temperature, and smart vaporizer device battery component strength, etc. The dosage calculation protocol can also use variable data such as unique quantitative formulation specifications of bioactive material. The dosage calculation protocol can perform (e.g., real-time) calculations of a user's intake of bioactive material during any single instance or specified duration of using vaporizer 101.

The dosage calculation protocol may also consider any of: user-specific health conditions, phenotypes, demographics, data from digital health tools and records, specifications of a bioactive material contained in bioactive material holding chamber 112, etc. Specifications of a bioactive material (including viscosity) can be accessed from bioactive material data 133, formulation data 134, etc. User specific data can be accessed directly from an external device or application or from user data 132.

Application 121 can implement a run/stop command protocol. The run/stop command protocol transmits run and stop commands (e.g., in control commands 142) from smart phone 120 to vaporizer 101, for example, using the data transfer protocol. Vaporizer 101 can receive a run/stop command over network link(s) 106. Vaporizer 101 can transfer the run/stop command to a processor in electronics 116. The processor converts the run/stop command to a corresponding on/off command. The on/off command is used to turn battery 117 on or off respectively. In one aspect, the on/off command is to battery 117 via an electrical current, which engages or disengages the power sent from battery 117 to other components (e.g., heating element 113).

On/off commands can be used to facilitate more precise control of a quantitative dosage, which is administered by stopping operation of vaporizer 101 after a designated dosage is administered (as determined, for example, by a quantitative molecular administering protocol. In response to an “on” control, power from battery 117 can be used at other components of vaporizer 101. In response to an “off” control, power from battery 117 is blocked from other components of vaporizer 101.

The run/stop protocol can utilize data from a (e.g., quantitative) dosage calculation protocol and a quantitative molecular administering protocol to calculate appropriate timing and duration for sending run/stop commands data to vaporizer 101.

Application 121 can implement a data encoded tag recognition protocol. The data encoded tag recognition protocol can include communication with an RFID transceiver included in electronics 116 to read a product recognition tag (e.g., RFID 121). The data encoded tag recognition protocol can include a translational decoding feature, which receives and identifies a unique data encoded identifier tag signal transmitted between vaporizer 101 and smartphone 102. The data encoded tag recognition protocol can initiate subroutines and other protocols tailored to the RFID data and quantitative bioactive material formulation data associated with data encoded cartridge 111.

As data encoded cartridge 111 is identified, the identification data (e.g., RFID 141) is acknowledged by other software application program protocols, allowing application 121 to (e.g., automatically) make decisions and calculations based on the unique bioactive material specifications which are contained in bioactive material holding chamber 112. The data encoded identifier tag recognition protocol can also operate as a geolocation tracking protocol, utilizing data related to the geolocation of data encoded cartridge 111 for compliance and raw material or product tracking. Further, the data encoded tag recognition protocol can also identify third-party manufactured smart vaporizer cartridges which have been manufactured using RFID smart-cartridge technology and recall quantitative composition data (when accessible from computations cluster 103 and/or smartphone 102).

Data encoder identifier tag and geolocation data can be linked to the GPS feature of smartphone 102 to enable restriction of device usage when GPS detects a user is in excess of normal walking speed (>5 MPH) or potentially operating a motor vehicle.

Application 121 can implement a quantitative molecular administering protocol. In general, the quantitative molecular administering protocol can utilize specifications of a bioactive material contained in bioactive material holding chamber 112 to calculate an appropriate (e.g., precision) dosage of bioactive molecules for a user. Specifications of a bioactive material can be accessed from bioactive material data 133, formulation data 134, etc.

The quantitative molecular administering protocol can perform real-time calculations pertaining to appropriate duration of “on” signals sent by the run/stop protocol to vaporizer 101. The quantitative molecular administering protocol can base calculations on specifications including but not limited to: battery 117 (power output to heating element), the temperature of heating element 113, a flowrate (e.g., ml/sec) of aerosolized bioactive material, the rate of molecular attrition/loss when bioactive materials contained within bioactive material holding chamber are aerosolized, etc. The quantitative molecular administering protocol can calculate and administer an appropriate (e.g., precise) dose of aerosolized bioactive material, by stopping operation of vaporizer 101 when a calculated bioactive material (designated dose) threshold has been administered. In one aspect, LED array 119 senses and optically measures flow rate approaching and/or having reached a determined threshold.

Application 121 can include a user interface permitting a user to make elections regarding and/or adjustments to his/her strength of dosage and his/her designated schedule for administering dosages. The quantitative molecular administering protocol can take into account user elections and adjustments as changes are submitted by a user or during manual operation of vaporizer 101 using LED-backed button 151.

The quantitative molecular administering protocol may also utilize specifications of a bioactive material contained in bioactive material holding chamber 112. Specifications of a bioactive material (including viscosity) can be accessed from bioactive material data 133, formulation data 134, etc.

The quantitative molecular administering protocol may also utilize user-specific biological or “omics” data obtained from the user, to administer a precision dosage prior to and during use of vaporizer 101.

As such, the quantitative molecular administering protocol can utilize quantitative molecular analysis associated with a registered formulation instance to deliver appropriate (e.g., precision) dosages of the bioactive material contained in bioactive material holding chamber 112. The quantitative molecular administering protocol can refer to predetermined calculations logged in computational cluster 103/database 131 to facilitate dose administration by communicating duration determinations to run/stop protocols (e.g., that control usage instance durations of smart vaporizer device use) and discrete battery 117 voltage determinations sent to heating element 113.

Logged calculations can be derived from data established during R&D and refer to verified data pertaining to one or more of: a) the temperature(s) reached by the heating element during activation, b) the rate at which any included bioactive material is aerosolized at temperatures conducted by the heating element(s), c) the rate of molecular attrition/loss of any bioactive materials used in any registered formulation instance, d) the detected, variable and/or the constant flow rate (measured as ml/sec) of the aerosolized bioactive delivered during administering with the smart vaporizer device and smart vaporizer cartridge, e) a precision measurement of the aerosolized bioactive material(s) output administered during usage instance(s) (measure in mg/sec), and f) a pressure at a which aerosolized bioactive material is being output.

Utilization of R&D established data can increase calculation precision of aerosolized bioactive material(s) output measured in mg/second or micrograms (μg nanogram)/second. Using these calculations, a quantitative molecular administering protocol can establish discrete voltage determinations and duration thresholds pertaining to the activation/deactivation and usage of vaporizer 101. The quantitative molecular administering protocol can recall data from computational cluster 103/database 131 that has established smart vaporizer device usage specifications durations utilized to administer appropriate (e.g., precision dosages) of an identified registered formulation instance of bioactive materials. Usage duration data can be communicated from the quantitative molecular administering protocol to run/stop protocols. The run/stop protocols can use the usage duration data to control activities of vaporizer 101 in accordance with the specified registered formulation instance data. Usage durations recalled from computational cluster 103/database 131 can vary at least in part upon variable formulations of bioactive material(s) contained in RFID embedded smart vaporizer cartridges and associated registered formulation instances.

In one aspect, portions of a quantitative molecular administering protocol interoperate with portions of a dosage calculation protocol to calculate an appropriate dose of aerosolized bioactive material for a user.

Application 121 can implement a queries and push notifications protocol. The queries and push notifications protocol provides a user of vaporizer 101 and application 121 scheduled administration reminders, poses pre- and post-consumption queries. The queries and push notifications protocol can permit a user to respond within application (e.g., through a user interface) to his/her subjective formulations and dosage response sentiments. The queries and push notifications protocol can permit a user to provide feedback related to the efficacy of an administered dose or series of dosages of the bioactive material formula from vaporizer 101.

The queries and push notifications protocol can be included in a UX/UI of application 121 and generates user-submitted data within application 121. The user-submitted data can then be transferred to database 131/computational cluster 103 (e.g., included in user data 132). Push notifications generated by the queries and push notifications protocol can pose user queries, such as, “prior to your dosage, how would you rate your symptom severity, on a scale of 1-10?”, and “after your dosage, how would you rate your symptom severity, on a scale of 1-10?”. Additional push notifications generated by the queries and push notifications protocol can provide user reminders to administer a scheduled dose of bioactive material, such as, “It's 8:30 am, and time to take your scheduled morning dose.”, and “Your evening dosage is 2 hours away, how are you feeling now?” Data/information from users' query responses is transferred from application 121 to database 131/computational cluster 103 via network links 106. Computational cluster 103 can perform continuous analytics on user data 132 as well as bioactive material 133, and formulation data 134 for product and system improvements as well as supervision.

Accordingly, the queries and push notifications protocol can be utilized to send queries, receive user feedback, send dosage schedule reminders, initiate purchasing from the user or server side, and communicate with vaporizer 101. Push notifications are messages that are “pushed” from computational cluster 103/database 131. For local notifications, application 121 can schedules dosage reminder notifications with an operating system of smart phone 102 and/or a user-specified calendar. Alternatively, application 121 generates a schedule reminder directly (e.g., if authorized to continuously run in the background).

When a dosage reminder's scheduled time is reached, or the event's programmed condition is met, a message is displayed in a user interface of application 121 to communicate a dosage or scheduled purchase reminder message. In general, such interactions and the data provided by users are transferred to computational cluster 103/database 131. Computational cluster 103/database 131 can perform sentiment analysis, subjective information evaluation, and machine learning strategies to augment formulations of bioactive materials and to improve application-generated recommendations, when feedback is suggestive of potential for formulation improvements.

Remote push notifications can be generated and managed by computational cluster 103/database 131. Application 121 can be registered with computational cluster 103/database 131, for example, with a unique key (e.g., a UUID). Computational cluster 103/database 131 can then send messages against the unique key to deliver the messages to application 121 via an agreed client/server protocol, such as HTTP or XMPP. Application 121 can display received messages.

When a push notification arrives, application 121 can transmit short notifications and messages, set badges on application icons, blink or continuously light up the notification LED, play alert sounds to attract the user's attention. Application 121 can also request feedback from the user pertaining to his/her experience with vaporizer 101, data encoded cartridge 111 (or other cartridges), formulations 134 of bioactive materials 133, or application 121. Message content can be classified in the following example categories: chat messages, vendor offers/specials, event (dosage timing/refill/etc.) reminders, and/or subscribed topics pertaining to any additional features of application 121 and the user's interactivity therein.

Adherence to data exchange and data/information collection can be incentivized with internal reward currency, wherein users generate incentives currency through engagements with one or more of: vaporizer 101, application 121, or computational cluster 103/database 131. Another push notification category can include pre/post-consumer data collection, wherein computational cluster 103/database 131 sends user-specific queries to extract feedback from users pertaining to their experiences with, and the biological effects of, a standardized or personalized formulation of bioactive material(s) contained in a data encoded smart vaporizer cartridge (e.g., in bioactive material holding chamber 112). Data/information can be collected, aggregated, filtered, and curated by computational cluster 103/database 131 and used in machine learning, adaptive dosage, and/or formulation improvement strategies.

Application 121 can implement a predictive dosage & refill reminder push notification protocol. The predictive dosage & refill reminder push notification protocol can provide users with real-time calculations, depletion predictions, and disclosures related to their intake of bioactive materials. The predictive dosage & refill reminder push notification protocol can also calculate the material remaining in bioactive material holding chamber 112 and provide reminders and direct pathways to refill or re-order new data encoded identifier tag enabled smart vaporizer cartridges & bioactive material.

The predictive dosage & refill reminder push notification protocol can send reminders prior to the calculated depletion predictions of smart vaporizer cartridge contents becoming critically depleted or emptied. The predictive dosage & refill reminder push notification protocol mitigates scenarios where a user is not mindful of the remaining contents of bioactive material holding chamber 112. As such, users are less likely to fully deplete bioactive material contained within bioactive material holding chamber 112 without having ordered a refill of needed bioactive materials.

Accordingly, the predictive dosage & refill reminder push notification protocol can continuously monitor usage of vaporizer 101 and data encoded cartridge 111. The predictive dosage & refill reminder push notification protocol can provide push notifications via application 121 when the bioactive material(s) in bioactive material holding chamber are nearing depletion or depleted. Push notifications can be generated and managed by computational cluster 103/database 131.

Data pertaining to: a) the temperature(s) reached by the heating element 113 during activation, b) the rate at which any included bioactive materials are vaporized at temperatures conducted by heating element 113, c) the rate of molecular attrition/loss of any bioactive materials used in any registered formulation instance, d) the detected, variable and/or the constant flow rate (measured as ml/sec) of the vaporized bioactive delivered during administering using vaporizer 101 and data encoded cartridge 111 (e.g., by LED array 119), and e) a measurement of the vaporized bioactive material(s) output administered during usage instance(s) (measure in mg/sec) can be logged by computational cluster 103/database 131. The data can be used to increase the precision of calculations related to vaporized bioactive material(s) output, measured in mg/second or micrograms (μg)/second.

Bioactive material(s) data can also be used to quantify the rate or stage at which the bioactive material is depleted. These analytics can be utilized by computational cluster 103/database 131 to render predictive models pertaining to the depletion rate of bioactive material in smart vaporizer cartridges. Predictive depletion models can contain content usage thresholds which, when approached or reached, notify computational cluster 103/database 131 of a bioactive material depletion stage and/or rate. In response, computational cluster 103/database 131 can generate a push notification, which is sent to application 121 (or web application platform), possibly triggering purchase/repurchase programming.

Purchase/repurchase programming provides users with a simple and streamlined system through which they may order/reorder the bioactive material(s) and/or smart vaporizer cartridges filled with bioactive material(s). The user receives the push notification which, when pushed, brings him/her to a purchasing page within application 121 (or web application platform). Using the purchasing page, the user can utilize a one-touch purchasing protocol to order or reorder the smart vaporizer cartridge and bioactive materials nearing depletion.

In some aspects, application 121 also includes one or more of: a product marketplace, telemedicine access to medical or holistic practitioners, clinical trial participation, educational resources, etc. Application 121 can also include functionality to answer ongoing detailed questionnaires to augment, revise, or change the formulation(s) of bioactive material(s) users receive.

Application 121 (and possibly also computational cluster 103) can import biomedical, biometric, bioinformatic, and other data sources. The data from these additional sources can be cross-referenced with the subjective user data, which can be utilized to develop personalized formulations of bioactive materials. Importing of subjective feedback data, along with data from additional data sources, supports an ongoing refinement of the bioactive materials formulation, and adaptive dosage strategy, which are continuously improved based on data received by and stored within application 121 and/or computational cluster 103/database 131. In general, effective combinations of bioactive material can be formulated through essentially real-time analysis of user needs and biological responses to provide effective therapeutic pathways and/or targeted results.

In some aspects, (e.g., a clinical version of) application 121 is HIPAA compliant. Application 121 can exchange HIPAA compliant data, informed consent (IRB) data, gather clinical data, interact with members of a clinical trial, demonstrate efficacy of bioactive material formulations, etc. In a clinical setting, application 121 can be configured with disease management protocols, telemedicine visit management, user-to-practitioner interaction, home health monitoring, user insurance reimbursement, prescription monitoring (e.g., medical cannabis, pharmaceuticals, and/or controlled substance(s))

Application 121 can also be implemented at a desktop web application platform.

Cluster

Computational cluster 103 and database 131 provide additional resources to and can perform computing on behalf of application 121. Data can be exchanged between application 121 and computational cluster 103 via network link(s) 106. Exchanged data can include formulation datasets related to the bioactive material (e.g., contained in bioactive material holding chamber 112), inventory/product identification information related to data encoded identifier tags (e.g., RFID 141) embedded on/within data encoded cartridge 111, and user-specific health data included in user data 132.

Data received at database 131 and/or computational cluster 103 can be continuously compiled and collated within user data 132, bioactive material data 133, formulation data 134, cartridge ID data 135, sensor data, etc. or other parts of database 131 for a variety of purposes. For example, data can be continuously compiled and collated for augmenting and improving adaptive dosage functions, research and formulation efficacy oversight, and micro-scale (instance) and macro-scale (network) user sentiment and vaporizer 101 usage analytics.

Feature controls programming within application 121 can also be adapted and generated from database 131 and/or computational cluster 103. Updates and programming changes to application 121 and any firmware updates for components in electronics 116 (e.g., a processor) can also be maintained at database 131 and/or computational cluster 103. In one aspect, data is transferred via cellular or WiFi connections.

In some aspects, application 121 and/or computational cluster 103 utilize geolocation and user/product verification protocols to ensure a data encoded cartridge is being used by its purchaser. Application 121 and/or computational cluster 103 can also implement anticounterfeiting measures. As cartridges are manufactured, the cartridges can be registered in cartridge ID data 135. When a cartridge is identified, application 121 and/or computational cluster 103 can verify/validate the cartridge using cartridge ID data 135.

Application 121 and/or computational cluster 103 can continuously monitor cartridge use, and through a predictive refill reminder protocol, identify when cartridges are depleted, resulting in an inability for counterfeiters to refill any emptied cartridge. Refilled cartridges can be detected by one or both of application 121 and computational cluster 103 as counterfeit. Application 121 can include an alert when a counterfeit cartridge is detected.

Mobile Device Configuration

Smartphone 102 (including a touch-control enabled screen) can download application 121. Application 121 can be NFC compliant for use with RFID identification protocols. Bluetooth can be used to “pair” application 121 with vaporizer 101. Application 121 can use a first one or more protocols to communicate with vaporizer 101 and a second different one or more protocols to communicate with computational cluster 103/database 131.

A user can use application 121 to create an account/profile including answering a preliminary questionnaire. The users account/profile data can be stored at smartphone 102 and/or in user data 132. Vaporizer 101 can be registered to the user's profile.

Vaporizer Configuration

In general, data encoded cartridge 111 includes a component, such as, a mouth piece, allowing a user to inhale aerosol from vaporizer 101 (e.g., via a mouth piece). To enable vaporizer 101 functionality, a user can enter an authorization code in the form of a designated alphanumeric passcode and/or biometric fingerprint scan. Authorization code entry can prevent unauthorized users, including children, from inhaling aerosol out of vaporizer 101.

Subsequent to authorization, data encoded cartridge 111 can be attached to (screwed onto) vaporizer 101. Data encoded cartridge 111 can be identified by vaporizer 101. Data encoded cartridge 111 can be “paired” with vaporizer 101 and/or application 121.

### Usage Example

A user can acquire smart vaporizer device and smart vaporizer cartridge(s), pre-loaded with bioactive material(s). The user then utilizes a compatible smartphone to download and install a software application. Once installed, the user then creates an account/profile, and answers a preliminary questionnaire. The user then establishes a pairing between the smartphone and smart vaporizer device, via Bluetooth connectivity of said devices. As this pairing (or “master/slave relationship”) is established, the software application is used to register the smart vaporizer device to the user's profile.

The user then completes smart vaporizer device authorization by entering the user's authentication data into the software application. The user then inserts a smart vaporizer cartridge into the smart vaporizer device. As this process is completed, the smart vaporizer device and software application identify a data encoded identifier tag embedded within the smart vaporizer cartridge.

The software application displays dosage options to the user, which are specific to the inserted smart cartridge and bioactive material therein, and user-specific data. As the smart cartridge is recognized by the system, the software application and a computational cluster recall the material composition of the registered bioactive material in the smart vaporizer cartridge. The software application and/or computational cluster_prepares commands to be sent to the smart vaporizer device. The commands can specify the appropriate temperature to optimize the aerosolization of the bioactive materials. The user then selects or approves a desired dosage, and the software application poses a query or series of queries prior to device activation.

Upon completing or bypassing these queries, the software application prepares the smart vaporizer for use. Activation of the smart vaporizer may be automatic and activated by beginning to pull air during the inhale process, or it may be activated through the pressing of an activator button (e.g., LED-backed button 151) on the device. To administer, the user then presses his lips around the mouthpiece of the smart vaporizer cartridge and begins to inhale to administer the aerosolized bioactive material vapor.

The software application provides users with real time data visualizations pertaining to the quantitative dosage which is administered. Upon reaching the intended dosage, the smart vaporizer device powers off, and the administering process is complete. At various durations post-administration, the software application sends push notifications to the user, inquiring as to the effects, efficacy, and other symptom related queries to gain subjective feedback which pertains to user's perception of his biological response and/or experience. Feedback can relate to the smart vaporizer device, smart vaporizer cartridge, software application, or another other feature listed in this invention or developed and included hereafter.

A model or calculations within the computational cluster can determine the smart vaporizer cartridge contents are nearing depletion, or have become depleted. In response, the software application generates a push notification which provides user(s) access to immediate recall of their previous purchase, as well as new purchase options, and the pathway to purchase refills of the bioactive materials or new smart vaporizer cartridges which are pre-filled with bioactive material(s).

Other Uses

Aspects of the invention can also be used in pharmaceutical applications, where aerosolized materials are inhaled by a user in a similar process. The smart vaporizer cartridges may also be made of materials or engineering which make the invention suitable for dry material vaporization, or powder delivery and inhalation. The programming/protocols found in the software application may be partially or entirely incorporated or programmed into the microprocessor of the smart vaporizer device, making the device a standalone device; a touchscreen and operating system may be incorporated into the smart vaporizer device to provide the components accommodating those functionalities.

Identifying Bioactive Material Holding Chamber Contents

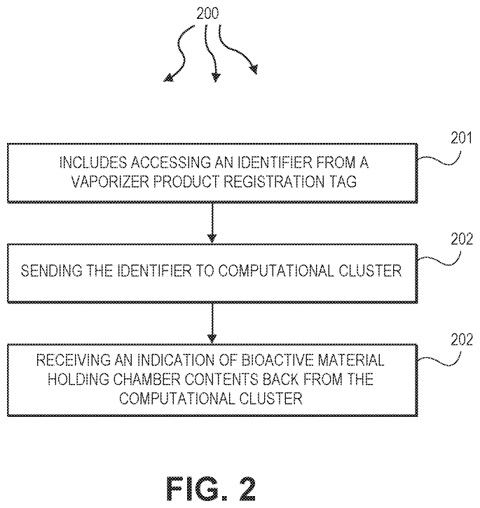

FIG. 2 illustrates a flow chart of an example method 200 for determining contents of a bioactive material holding chamber. Method 200 will be described with respect to the components and data in computer architecture 100.

Method 200 includes accessing an identifier from a vaporizer product registration tag (201). For example, application 121 can access identifier 141 associated with data encoded cartridge 111.

Method 200 includes sending the identifier to computational cluster (202). For example, application 121 can send identifier 141 to computational cluster 103. Computation cluster can utilize identifier 141 to query database 131. The query can return cartridge ID data from cartridge ID data 135, for example, data loaded into database 131 during manufacture of data encoded cartridge 111. Based on the cartridge ID data, computational cluster 103 can further refer to bioactive material 133 and formulation data 134 to derive the contents 143 bioactive material chamber 112. Computational cluster 103 can send an indication of contents 143 back to application 121.

Method 200 includes receiving an indication of bioactive material holding chamber contents back from the computational cluster (203). For example, application 121 can receive the indication of contents 143 from computational cluster 103.

Bioactive Material Delivery

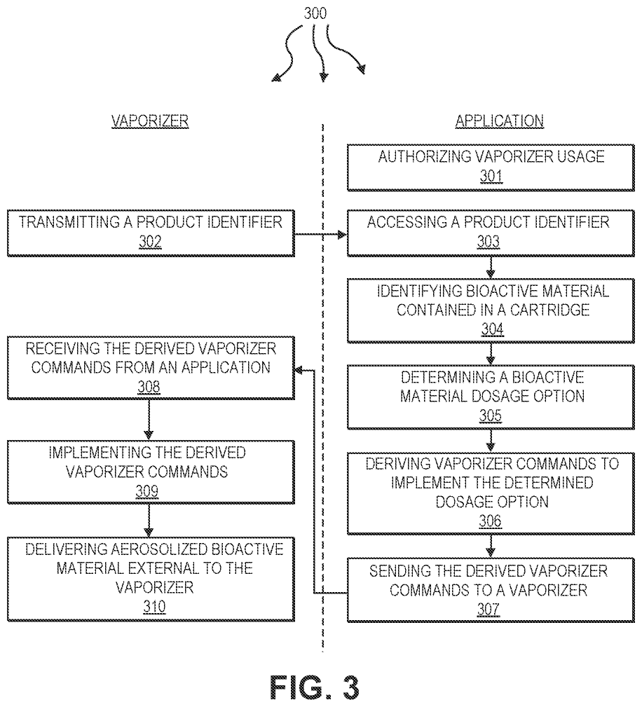

FIG. 3 illustrates a flow chart of an example method 300 for controlling bioactive material delivery. Method 300 will be described with respect to the components and data in computer architecture 100.

In some aspects, vaporizer 101 and application 121 are “paired” with one another prior to bioactive material delivery.

In general, application 121 and/or computational cluster 103 can control administrating doses of aerosolized bioactive material at vaporizer 101 in accordance with a quantitative dosage calculation protocol and/or a quantitative molecular administering protocol. In some aspects, method 300 is included in, integrated into, or interoperates with a quantitative dosage calculation protocol and/or a quantitative molecular administering protocol.

Method 300 includes authorizing vaporizer usage (301). For example, a user can enter a passcode from vaporizer 101 at application 121. Application 121 can verify that the passcode matches a previously selected passcode associated with vaporizer 101.

Method 300 includes transmitting a product identifier (302). For example, subsequent to attachment to vaporizer 101, data encoded cartridge 111 can transmit RFID 141 to smartphone 102. Method 300 includes accessing a product identifier (303). For example, smartphone 102 can received RFID 141 from vaporizer 101. Application 121 can access RFID 141 at smartphone 102. In one aspect, data encoded cartridge 111 is attached to vaporizer 101 after user authorization. Upon attachment, a product recognition tag at data encoded cartridge 111 can be energized by battery 117 and can emit RFID 141. A component at vaporizer 101 can detect RFID 141 and send RFID 141 to smartphone 102. Alternatively, a component at smartphone 102 can detect RFID 141 directly.

Method 300 includes identifying bioactive material contained in a cartridge (304). For example, application 121 can identify bioactive material contained in data encoded cartridge 111. In one aspect, application 121 sends RFID 141 (e.g., in a query) to computational cluster 103. Computational cluster 103 identifies the composition of bioactive material corresponding to RFID 141 (and thus contained in data encoded cartridge 111) from within database 131. Computational cluster 103 can indicate the identified composition of bioactive material (e.g., in 143) back to application 121

Method 300 incudes determining a bioactive material dosage option (305). For example, application 121 (and/or computational cluster 103) can determine a dose of bioactive material contained in data encoded cartridge 111. In one aspect, application 121 (and/or computational cluster 103) determines an appropriate dose of bioactive material based on user data corresponding to a user (e.g., in user data 132) and bioactive material data corresponding to the bioactive material (e.g., in bioactive material data 133). Application 121 (and/or computational cluster 103) can also determine an appropriate dose of bioactive material based on: other data from vaporizer 101, other data stored at application 121, or other data in database 131 (e.g., formulation data 134).

Application 121 (and/or computational cluster 103) can utilize any of the described algorithms to determine one or more dosage options for an identified bioactive material. In one aspect, a bioactive material dosage option is determined automatically. In another aspect, a bioactive material dosage option is determined based on user input. In a further aspect, a bioactive material dosage option is determined through interoperation of automated algorithms and human input.

It may be that a plurality of different possible bioactive material doses or dosing options are determined using automated algorithms and/or based on human input.

In one aspect, application 121 presents any determined bioactive material doses or dosing options (for the contents of data encoded cartridge 111) on a (e.g., touch screen) display (e.g., in a user interface) at smartphone 102. A user can then select a bioactive material dose or dosing option from among the presented bioactive material doses or dosing options (e.g., through the user interface).

In other aspects, determining a bioactive material dossing option is fully automated. For example, application 121 and/or computational cluster 103 can determine a bioactive material dose without user input (and/or without even presenting the dosing option to a user).

Method 300 includes deriving vaporizer commands to implement the determined dosage option (306). For example, application 121 can derive commands to implement the determined dosage option at vaporizer 101. Application 121 can calculate one or more of a specified heating element temperature and a specified cartridge pressure appropriate for the selected dosage option. Application 121 can then formulate commands to turn on vaporizer 101, to adjust heating element 113 to the specified temperature, and adjust data encoded cartridge 111 to the specified pressure. Application 121 can package the commands in commands 142. Method 300 includes sending the derived vaporizer commands to a vaporizer (307). For example, application 121 can send commands 142 to vaporizer 101.

Method 300 includes receiving the derived vaporizer commands form an application (308). For example, vaporizer 101 can receive commands 142 from application 121.

In general, vaporizer 101 can implement received vaporizer commands to configure components of vaporizer 101 to aerosolize bioactive material contained in bioactive material holding chamber 112. In general, vaporizer 101 can also implement received vaporizer commands to operate components of vaporizer 101 aerosolizing bioactive material contained in bioactive material holding chamber 112 and delivering aerosolized bioactive material out of mouthpiece 139.

Method 300 includes implementing the derived vaporizer commands (309). For example, a processor in electronics 116 can implement the derived vaporizer commands at vaporizer 101. The processor can turn on battery 117, allow specified current to flow from battery 117 to heating element 113 to adjust heating element 113 to the specified temperature, and allow current to flow to pressure control mechanism 114 to adjust pressure in data encoded carriage 111 to the specified pressure.

Method 300 includes delivering aerosolized bioactive material external to the vaporizer (e.g., in accordance with the determined dosage option) (310). For example, heating element 113 can aerosolize bioactive material from bioactive material holding chamber 112. Aerosolized bioactive material can be delivered out mouthpiece 139, which potentially includes LED array 119, at a specified pressure. In one aspect, sensors monitor temperature and pressure. When the specified temperature and pressure are achieved at vaporizer 101, vaporizer 101 can send a ready message to application 121. Application message 121 can indicate that vaporizer 101 is ready for use (e.g., through a user interface) at smartphone 102. Alternatively, or in combination, vaporizer 101 may include LED indicators (e.g., in LED-backed button 151). The processor can switch or transition the LED indicators from one color to another color, for example, from red to green, when the specified temperature and pressure are achieved.

A user can then activate aerosolization and inhale aerosol from vaporizer 101. Activating vaporizer 101 may be automatic and activated by beginning to pull air during the inhale process on a mouth piece of data encoded cartridge 111, or it may be activated through the pressing of an activator button (e.g., LED-backed button 151) on vaporizer 101. To administer, the user then presses his lips around the mouthpiece e and begins to inhale to administer the bioactive material aerosol. Sensors at vaporizer 101, for example, LED array 119, can monitor and/or measure the volume of bioactive material aerosol being administered over time, either between or duration inhalations. The sensors can indicate the bioactive material aerosol volume to application 121 in essentially real-time.

Application 121 can provide real-time visualizations representing the volume of an administered dose. Upon reaching an intended dosage, application 121 can formulate further vaporizer commands to turn off vaporizer 101. Application 121 can package the commands in further commands 142. Application 121 can send the further commands 142 to vaporizer 101.

Vaporizer 101 can receive the further commands 142 from application 121. The processor in electronics 116 can implement the further vaporizer commands at vaporizer 101. The processor can turn off battery 117 cutting power to heating element 113 and pressure control mechanism 114. Application message 121 can indicate that vaporizer 101 is turned off (e.g., through a user interface) at smartphone 102. Alternatively, or in combination, the processor can switch or transition the LED indicators from one color to another color, for example, from green to red, when power is cut.

Subsequent to administering a dose, method 300 can be repeated to administer another dose in accordance with a quantitative dosage calculation protocol and/or a quantitative molecular administering protocol. Dose administration for a user can be tracked over time, including tracking bioactive material type, bioactive material volume, date/time of last dose, etc. In one aspect, dosage schedule is followed. Vaporizer 101 can be activated from time to time, with a specified frequency, or at specified intervals in accordance with the dosage schedule.

As such, application 121 can control vaporizer 101 to administer a tailored dose (or doses) of aerosolized bioactive material to a user of vaporizer 101 over time. Doses of aerosolized bioactive material can be tailored using a quantitative dosage calculation protocol and/or a quantitative molecular administering protocol based on vaporizer 101 operating characteristics (e.g., temperature of heating element, flow rate (e.g., ml/sec), etc.), and possibly along with one or more of: user data 132, bioactive material data 133, formulation data 143, or cartridge ID data 135.

At various durations post-administration, application 121 can send push notifications to the user, inquiring as to the effects, efficacy, and other symptom related queries to gain subjective feedback. The feedback can pertain to user's perception of his experience with vaporizer 101, date encoded cartridge 111, application 121 or other aspects of the invention.

Push notification can include notifying a user when contents of bioactive material chamber 112 are approaching depletion. The push notification can recall a previous purchase, as well as new purchase options, and the pathway to purchase refills of the bioactive materials or new smart vaporizer cartridges which are pre-filled with bioactive material(s).

Aspects of the invention can be used in pharmaceutical applications, where aerosolized materials are inhaled by a user in a similar process. For example, bioactive material holding chamber can contain-over-the counter or prescription medications. Data coded cartridges can be made of materials suitable for dry material or powder inhalation.

In some aspects, the programming/protocols described with respect to application 121 can be partially or entirely incorporated or programmed into a processor of a (e.g., standalone) smart vaporizer device. A touchscreen and operating system may be incorporated into the smart vaporizer device to provide the components accommodating those functionalities. As such, a smart vaporizer device can communicate directly with computation cluster 103/database 131 and implement aspects of the invention associated with application 121. For example, a smart vaporizer can implement any of: a data transfer protocol, a device authorization and user/child safety protocol, GPS-driven motion detection and device usage restrictions, a quantitative dosage calculation protocol, a run/stop command protocol, a data encoded tag recognition protocol, a quantitative molecular administering protocol, a queries and push notifications protocol, a predictive dosage and refill reminder push notification protocols, etc.

In other aspects, portions of the various described protocols are distributed across a smart vaporizer device (e.g., vaporizer 101) and an application running at another processing device (e.g., application 121 running on smartphone 102). In further aspects, portions of the various described protocols are distributed across a smart vaporizer device (e.g., vaporizer 101), an application running at another processing device (e.g., application 121 running on smartphone 102), and a computer cluster/database (e.g., computational cluster 103/database 131).

The present described aspects may be implemented in other specific forms without departing from its spirit or essential characteristics. The described aspects are to be considered in all respects only as illustrative and not restrictive. The scope is, therefore, indicated by the appended claims rather than by the foregoing description. All changes which come within the meaning and range of equivalency of the claims are to be embraced within their scope.

What is claimed:

1. A method comprising:
authorizing use of a vaporizer by a user;
receiving a cartridge product identifier from the vaporizer;
identifying bioactive material contained in a vaporizer cartridge based on the cartridge product identifier;
determining a bioactive material dosage option based at least on user data corresponding to the user and bioactive material data corresponding to the identified bioactive material;
deriving vaporizer control commands to implement the formulated bioactive material dosage option; and
sending the vaporizer control commands to the vaporizer.

2. The method of claim 1, wherein authorizing use of a vaporizer comprises validating a passcode associated with the user or validating biometric information of the user.

3. The method of claim 1, wherein identifying bioactive material contained in a vaporizer cartridge based on the cartridge product identifier comprises:
querying a computational cluster database with the cartridge product identifier;
receiving an indication of a bioactive material back from the computational cluster database.

4. The method of claim 1, wherein determining a bioactive material dosage option comprises receiving a user selection of the bioactive material dose.

5. The method of claim 1, wherein determining a bioactive material dosage option comprises tailoring a bioactive material dose based on effects of prior doses of the bioactive material on the user.

6. The method of claim 1, wherein deriving vaporizer control commands comprises deriving vaporizer control commands including one or more of: a vaporizer battery control command, a vaporizer heating element control command, or a vaporizer pressure valve control command; and
wherein sending the vaporizer control commands to the vaporizer comprises sending the one or more of: the vaporizer battery control command, the vaporizer heating element control command, or the vaporizer pressure valve control command.

7. The method of claim 1, further comprising:
receiving an indication of an aerosol delivery rate at the vaporizer;
estimating when a bioactive material dose is fully delivered at the vaporizer based on the aerosol delivery rate; and
sending a stop control command to the vaporizer when the bioactive material dose is estimated to have been fully delivered.

8. The method of claim 1, further comprising receiving user feedback associated with effects of a bioactive material dose on the user.

9. The method of claim 1, further comprising calculating depletion of the bioactive material in the cartridge based the amount of bioactive material in the determined bioactive material dosage option and prior bioactive material doses administrated from the cartridge in view of a total amount of bioactive material originally included the cartridge; and
sending a reminder push notification to purchase a new cartridge of the bioactive material.

10. A method comprising:
electronically accessing a cartridge identifier from a vaporizer cartridge;
sending the cartridge identifier to an application;
receiving authorization from the application to vaporize bioactive material contained in a cartridge;
receiving vaporizer control commands from the application and defining a determined bioactive material dose; and
implementing the received vaporizer commands, including:
configuring vaporizer components to aerosolize bioactive material contained in a cartridge; and
operating the vaporizer components delivering aerosolized bioactive material in accordance with the determined bioactive material dose.

11. The method of claim 10, wherein electronically accessing a cartridge identifier comprises: energizing a Radio Frequency (RF) tag and detecting a Radio Frequency Identified (RFID) emitted by the RF tag.

12. The method of claim 11, wherein sending the cartridge identifier to an application comprises sending the RFID to the application.

13. The method of claim 10, wherein receiving authorization from the application to vaporize bioactive material contained in a cartridge comprises:
sending a biometric information to the application; and
receiving an indication from the application that the biometric information is valid.

14. The method of claim 10, wherein receiving authorization from the application to vaporize bioactive material contained in a cartridge comprises receiving as command enabling a battery to power other vaporizer components.

15. The method of claim 10, wherein receiving vaporizer control commands comprises receiving one or more of: a battery enablement command, a heating element temperature command, or a pressure setting command.

16. The method of claim 15, wherein configuring vaporizer components to aerosolize bioactive material contained in a cartridge comprises implementing one or more of: the battery enablement command, the heating element temperature command, or the pressure setting command.

17. The method of claim 10, further comprising monitoring the amount of bioactive material aerosol exiting a mouth piece and restricting further vaporizer usage when the amount of bioactive material aerosol reaches the formulated bioactive material dose.

18. The method of claim 10, further comprising:
storing vaporizer usage data locally at the vaporizer; and
sending the vaporizer usage data to the application when a network connection between the vaporizer and the application is detected
